(19) United States
(12) Patent Application Publication
(10) Pub. No.: US 2024/0066142 A1
(43) Pub. Date: Feb. 29, 2024

(54) A UPAR (UROKINASE PLASMINOGEN ACTIVATOR RECEPTOR)-TARGETING CONJUGATE

(71) Applicant: FLUOGUIDE A/S, COPENHAGEN N (DK)

(72) Inventors: Line HARTVIG, HORSHOLM (DK); Grethe NORSKOV RASMUSSEN, FARUM (DK); Morten ALBRECHTSEN, CHARLOTTENLUND (DK)

(21) Appl. No.: 18/272,210

(22) PCT Filed: Jan. 14, 2022
(86) PCT No.: PCT/EP2022/050715

§ 371 Date: Jul. 13, 2023

(30) Foreign Application Priority Data
Jan. 15, 2021 (SE) 2150038-4

(51) Int. Cl. A61K 47/68; A61K 49/00

(52) U.S. Cl. CPC A61K 47/6851; A61K 49/0056

(57) ABSTRACT

The present invention describes a uPAR (urokinase Plasminogen Activator Receptor)-targeting conjugate comprising: —a fluorophore; —a molecule binding to the receptor; and—a linker group which covalently links the fluorophore to the molecule binding to the receptor, said linker group either being part of the molecule binding to the receptor or being a separate component of the uPAR (urokinase Plasminogen Activator Receptor)-targeting conjugate; wherein the uPAR (urokinase Plasminogen Activator Receptor)-targeting conjugate has a pharmacokinetic profile where a human TBR (tumor-to-background ratio) of at least 1.1 is reached within 3.5 hours post administration and where a human TBR of at least 1.5 is maintained at least 30 minutes before decreasing below 1.5, and wherein the uPAR-targeting conjugate is a human uPAR-targeting conjugate.

### FIELD OF THE INVENTION

The present invention relates to a uPAR (urokinase Plasminogen Activator Receptor)-targeting conjugate with an improved pharmacokinetic profile.

### TECHNICAL BACKGROUND

There are known receptor-targeting conjugates intended for administration in a human or animal body. For instance, in WO 2016/041558 there is provided a conjugate composition that binds to the cell surface expressed uPAR protein. The conjugate is based on a fluorescence-labelled peptide useful as a diagnostic probe which binds to the surfaces of cells expressing uPAR. The conjugate carries a suitable detectable and visible label that will allow qualitative detection of uPAR expressing cells in vitro and in vivo. This renders the surgical resection of tumors more optimal.

One aim of the present invention is to provide a uPAR (urokinase Plasminogen Activator Receptor)-targeting conjugate with an improved pharmacokinetic profile in relation to what has been provided in the past in corresponding uPAR (urokinase Plasminogen Activator Receptor)-targeting conjugates.

### SUMMARY OF THE INVENTION

The present invention is directed to a uPAR (urokinase Plasminogen Activator Receptor)-targeting conjugate comprising:
a fluorophore;
a molecule binding to the receptor; and
a linker group which covalently links the fluorophore to the molecule binding to the receptor, said linker group either being part of the molecule binding to the receptor or being a separate component of the uPAR (urokinase Plasminogen Activator Receptor)-targeting conjugate; wherein the uPAR (urokinase Plasminogen Activator Receptor)-targeting conjugate has a pharmacokinetic profile where a human TBR (tumor-to-background ratio) of at least 1.1 is reached within 3.5 hours post administration and where a human TBR of at least 1.5 is maintained at least 30 minutes before decreasing below 1.5,
   and wherein the uPAR-targeting conjugate is a human uPAR-targeting conjugate.

In relation to the above it may be mentioned that the conjugate shown in WO 2016/041558 is not directed to providing an optimal pharmacokinetic such as according to the present invention and as is disclosed above.

### Description and Embodiments of the Invention

The present invention relates to a human uPAR-targeting conjugate with an adequate receptor binding profile in combination with an optimal pharmacokinetic profile intended for administration in a human body.

As mentioned above, the uPAR-targeting conjugate has an optimal receptor binding affinity and pharmacokinetic profile. The uPAR-targeting conjugate injected systemically will distribute through the circulatory system to blood perfused tissues and organs in the body. For tissues with blood perfusion the uPAR-targeting conjugate will accumulate. When such tissue is exposed to light with a wavelength (color) being absorbed by the fluorophore contained in the uPAR-targeting conjugate, the fluorophore will emit light. The uPAR-targeting conjugate (‘L’ in the formula below) binds to the receptor (‘R’ in the formula below) according to first order kinetics:

R+L [symbol] RL, and the reaction is characterized by the on-rate binding (Kₒₙ), the off-rate binding (K_{off}) and the resulting equilibrium binding constant K_{D} (K_{D}=K_{off}/Kₒₙ).

The uPAR-targeting conjugate will distribute via the blood from where it will be eliminated via excretion by the liver, and/or the kidney, or by redistributes to compartments other than the blood. It results in the tissue, with the presence of cells expressing the targeted receptor to where the uPAR-targeting conjugate is bound, lighting up more than the background, creating the so called “TBR” (tumor-to-background ratio). The conjugate is lighted up using a light source creating light of a specific wavelength and then detecting the light emitted from fluorophore using a specific filter for the specific emitted light. In a thought ideal situation, the cancer cells will light up immediately after injection, with sufficient high relative light intensity, without any background light, creating the desired TBR in an animal model of at least 2.5 lasting several hours. As can be noted, the values of human TBR and animal TBR are not the same. A threshold for an animal TBR of around 2.5 corresponds to a human TBR of about 1.5 (see e.g. Theranostics 2018, Vol. 8, Issue 19 in “Recommendations for reporting on emerging optical imaging agents to promote clinical approval”, Willemieke S. Tummers et al.). In the following and above there is specified if the TBR value is provided based on human TBR or animal TBR. The reasons for the above being: (1) The animal (e.g. mouse) is physically smaller than a human and there will be less noise in the fluorescent signal in animals than humans, and (2) the uPAR difference between animals and man, where a typical human uPAR targeted probe does not bind to animal uPAR, which means that the background binding in an orthotopic mouse model will have human uPAR expressed in the cancer as it is derived from human, where the normal mouse tissue being the background will not have human uPAR expressed and hence not binding a human uPAR probe.

Moreover, as should be understood from above and below, TBR is a well-known and widely used parameter in this field. One suitable definition and explanation of TBR is found in Maxwell Chamberlain Memorial Paper for General Thoracic Surgery, “Multiinstitutional Phase 2 Clinical Trial of Intraoperative Molecular Imaging of Lung Cancer, Sidhu Gangadharan, MD, et al.

In line with the above, the present invention is directed to a uPAR-targeting conjugate with a pharmacokinetic profile where an animal TBR (tumor-to-background ratio) of at least 2.5 is reached within 3.5 hours post administration and where an animal TBR of at least 2.5 is maintained at least 30 minutes before decreasing.

Moreover, also other parameters are of relevance according to the present invention. One such is the size of the uPAR-targeting conjugate, others are parameters directly related to pharmacokinetics, such as human half-life and plasma half-life.

According to one embodiment of the present invention, the uPAR-targeting conjugate has a size of less than 50 kDa, preferably less than 20 kDa, more preferably less than 5 kDa. In this regard it may be mentioned that typical antibodies fall within a size range of 25-50 kDa and a typical Fab is in a range of 8-16 kDa. As should be understood, all of these different groups are possible to provide according to the present invention, however a typical focus is within the mid-size range to small sizes, i.e. below 5 kDa. Smaller sizes are preferred as this increases tissue penetration when being used for medical purposes.

An optimal combination of size and the pharmacokinetic profile as such is also of relevance according to the present invention. According to one embodiment of the present invention, the uPAR-targeting conjugate has a size of less than 50 kDa, preferably less than 20 kDa, more preferably less than 5 kDa; and wherein the uPAR-targeting conjugate has a biological half-life in a human of at least 1 h. As will be understood from below, according to one preferred embodiment of the present invention the uPAR-targeting conjugate has a biological half-life in a human of at least 1 hour and maximum 75 hours.

Furthermore, according to one embodiment, the uPAR-targeting conjugate has a pharmacokinetic profile where the plasma half-life is maximum 75 hours, preferably maximum 20 hours, more preferably maximum hours, more preferably in the range of 6-15 hours, most preferably in the range of 6-10 hours.

Moreover, according to yet another embodiment, the uPAR-targeting conjugate has a pharmacokinetic profile where an animal TBR of at least 2.8 is reached within 3.5 hours post administration and where an animal TBR of at least 2.8 is maintained at least 30 minutes before decreasing.

Furthermore, also the peak value of the TBR may be of relevance. In this context it may be mentioned that according to one embodiment of the present invention, a peak animal TBR of the uPAR-targeting conjugate after administration is at least 3.

Moreover, the human uPAR-targeting conjugate according to the present invention exhibits several features including some linked to its pharmacokinetic and receptor binding affinity. The human uPAR-targeting conjugate provides a specific combination of plasma half-life and receptor binding affinity.

The TBR is calculated from the relative intensity of light from tumor and background. The measurement of the light intensity may e.g. be performed by using a simple commercially available camera with physical filters, such as, but not limited to, the clinically approved NIR-camera system, e.g. Fluobeam®800 (Fluoptics, Grenoble, France) or EleVision™ (Medtronic, USA). Post image recording optimization of the image using software may be applied to enhance the TBR.

According to the present invention, the administration is done systemically, preferably intravenously why the plasma concentration quickly reaches its highest concentration. The plasma concentration will decrease thereafter as the uPAR-targeting conjugate is eliminated via liver and/or kidney or distributes to distribution compartment differently to the blood. According to the present invention, the concentration shall be sufficiently high and be maintained for a sufficiently long period for the uPAR-targeting conjugate to reach a high enough concentration in the compartment relevant for the receptor binding (e.g. plasma, tissue stoma, cerebrospinal fluid, urine). According to one specific embodiment of the present invention, the dosing is performed in the range of 0.1-2,000 mg product per human. According to one embodiment, the dosing is performed in the range of 1-50 mg product per human patient. The uPAR-targeting conjugate according to the present invention can be dosed in a fixed dose per patient and does not have to be dosed relative to the patient weight.

Moreover, also sensitivity for cancer is of interest in relation to the present invention. According to one specific embodiment of the present invention, the uPAR receptor-targeting conjugate has a sensitivity for detection of cancer tissue of at least 60%, preferably above 70%, more preferably above 80% and most preferably above 90%. With sensitivity for detection of cancer tissue is understood the probability of a positive test result of the tissue sample (light up) when the tissue sample contains disease evaluated microscopically judged by a pathologist. An example is that the surgeon removed 10 tissues samples that he/she believes is cancer and seven of them is confirmed histologically is cancer given a selectivity of 7/10 (70%). If 100% of the tissue samples removed by the surgeon believing is cancer are proven to be cancer, the sensitivity is 100%.

One other aspect in relation to the aspects above are the place of excretion and elimination. Different conjugate types according to the present invention excretes and/or eliminates in different organs and are as such not suitable for cancer types localized in these organs. Moreover, the type of indication and target receptor are also important according to the present invention. The preference here is that the receptor needs to be expressed on the cancer the patient has to the maximal benefit for the operator (e.g. the surgeon). Furthermore, it is of specific interest that the receptor is expressed on the right part of the cancer. This is of interest as normally the middle of the cancer is easy to see and remove by the surgeon. The borders and local invasive outgrowths from the cancer, however, are more difficult to see and separate from normal tissue by the surgeon, hence more difficult for the surgeon to remove and/or to save normal tissue.

Ideally the uPAR receptor-targeting conjugate product is administered to the patient when the surgeon undertakes the surgical procedure of removing the cancer, including when the surgeon investigates the completeness of the surgical procedure by investigating the removed cancer tissue and by investigating if there are any cancer cells left in the patient right after having removed the cancer tissue, investigate the tissue removed or when planning the post-surgery treatment. This is e.g. immediately before or under the surgery, or right after, during or before the anesthesia. This is a huge improvement in comparison with other known alternatives which must be administered 6 hours, or even 1-2 day in advance of surgery to be ready for use during surgery and not even in every case produce a satisfactory TBR or having a satisfactory sensitivity or specificity. The combination of the features of receptor binding and plasma clearance according to the present invention will allow such improved use. In other words, the uPAR-targeting conjugate according to the present invention has a pharmacokinetic profile that allows administration close to the time of surgery, e.g. during the initiation of the anesthesia. It may furthermore be said that the uPAR-targeting conjugate according to the present invention enables that the time from administration to first feasible time for use is within at least 2,000 minutes, 300 minutes, or 120 minutes, such as preferably within 60 minutes, or even within 30 minutes, e.g. within 15 minutes from administration.

As is evident from above, the uPAR-targeting conjugate comprises a fluorophore; a molecule binding to the receptor; and a linker group which covalently links the fluorophore to the molecule binding to the receptor. The fluorophore may be of different type according to the present invention. According to one embodiment of the present invention the fluorophore is a near-infrared I fluorophore or a near-infrared II fluorophore. Interesting examples are fluorophores selected from the group consisting of ICG, Methylene blue, 5-ALA, Protoporphyrin IX, IRDye800CW, ZW800-1, Cy5, Cy7, Cy5.5, Cy7.5, IRDye700DX, Alexa fluor 488, Fluorescein isothiocyanate. According to another specific embodiment of the present invention the fluorophore may be a fluorophore selected from the group consisting of Flav7, CH1055, Q1, Q4, H1, IR-FEP, IR-BBEP, IR-E1, IR-FGP, IR-FTAP.

Moreover, a “linker group” is a molecule that connects the two parts to create the conjugate and where their respective functions to a large degree is maintained, e.g. where a peptide binds to a receptor and a fluorophore lights up. The linker connects the two together (the peptide and the fluorophore) where their desired properties are preserved in total or partially. This means e.g. that the peptide still binds to the receptor and the fluorophore preserves its properties. A linker can be at least partly part of either of the two molecules linked.

Moreover, the peptide may also of different type. According to one specific embodiment of the present invention, the peptide comprises or is selected from:
Asp-Cha-Phe-D-Ser-D-Arg-Tyr-Leu-Trp-Ser(−);
Asp-Cha-Phe-D-Ser-D-Arg-Tyr-Leu-Trp-Ser-OH; or
Asp-Cha-Phe-D-Ser-D-Arg-Tyr-Leu-Trp-Ser-NH₂.

To give some examples, the following may be mentioned: AE105 with the sequence DChaFsrYLWS-OH, AE344 with the sequence EE-O2Oc-O2Oc-DChaFsrYLWS-OH, AE345 with the sequence EE-O2Oc-O2Oc-DChaFsrYLWS-NH2, AE346 with the sequence O2Oc-O2Oc-DChaFsrYLWS-OH, AE347 with the sequence EE-O2Oc-DChaFsrYLWS-NH2, AE348 with the sequence E-O2Oc-DChaFsrYLWS-NH2, AE349 with the sequence EE-DChaFsrYLWS-OH, the sequence ICG-EE-DChaFsrYLWS-OH or AE353 with the sequence IRDye800CW-EE-020c-020c-DChaFsrYLWS-OH.

Furthermore, the amino acid may be selected from proteinogenic amino acids and non-proteinogenic amino acids, which includes natural amino acids and synthetic amino acids. In relation to this, it may further be mentioned that the natural amino acids may include C-alpha alkylated amino acids such aminoisobutyric acid (Aib), N-alkylated amino acids such as sarcosine, and naturally occurring beta-amino acids such as beta-alanine. Further, the synthetic amino acids may include amino acids with non-proteinogenic side-chains such as cyclohexyl alanine, gamma-amino acids, and dipeptide mimics. The term dipeptide mimics may be interpreted as an organic molecule that mimics a dipeptide by displaying the two amino acid side-chains, e.g., having a reduced amide bond linking two residues together. Amino acids with non-proteinogenic side-chains may also include amino acids with side-chains with restricted motion in chi-space. The term restricted motion in chi-space may be interpreted as restricted flexibility in the rotation of the side-chain groups. The oligopeptides may consist of up to fifty amino acids and may include dipeptides, tripeptides, tetrapeptides, and pentapeptides, and may further be made up by proteinogenic amino acids and non-proteinogenic amino acids.

Furthermore, the present invention also refers to a pharmaceutical composition comprising the uPAR-targeting conjugate according to the present invention together with at least one pharmaceutically acceptable carrier or excipient.

Further Embodiments of the Invention, Expressions, Data with Figures and Measurements According to the Present Invention

### Binding Affinity

K_{D} is determined by the on-binding kinetic measured as Kₒₙ and off-binding kinetic measured as K_{off}. K_{D} is the ratio between K_{off} and Kai. The lower the K_{D} the higher the binding affinity.

Although, an uPAR-targeting conjugate is developed under the drug development regulation and practices, the aim is lightly different. An uPAR-targeting conjugate is bound to the receptor (uPAR) to light up the cancer tissue. It is desired that once the uPAR-targeting conjugate is dosed, the uPAR-targeting conjugate rapidly reaches the cancer target organ and binds to the receptor while the remaining conjugate is washed away rather quickly to create a high TBR. This will enable the surgeon to perform the tumor removal soon after dosing.

An ideal uPAR-targeting conjugate should therefore have an optimal binding profile as well as pharmacokinetic profile. The Kₒₙ should, in particular, be as high as possible, and at least as high as the natural protein (uPA) which the uPAR-targeting conjugate competes with. It means in practice that a uPAR-targeting conjugate with a higher likelihood will bind to a free uPAR than the natural protein (uPA), assuming everything else is equal.

When the Kₒₙ is very fast, as is the case with the two examples according to the present invention.

The speed of which the protein-ligand complex takes place, and its life span is important in relation to the present invention.

As mentioned above, the uPAR-targeting conjugate (L) binds to the receptor (uPAR, R) according to R+L [symbol] RL.

Kₒₙ is the constant of the binding reaction. Its units are M⁻¹×s⁻¹. K_{off} is the constant for the dissociation of the ligand from the receptor. The dimension of K_{off} is time⁻¹. K_{D} is the equilibrium constant for the dissociation equilibrium equal to K_{off}/Kₒₙ (measured in M).

With reference to the present invention a certain characteristic is needed for the ligand receptor binding.

According to one specific embodiment of the present invention Kₒₙ is >1×10³ M⁻¹ s⁻¹, preferentially >1×10⁵ M⁻¹ s⁻¹. Moreover, according to yet another specific embodiment of the present invention, K_{off} is <1×10⁻¹ s⁻¹, preferentially <1×10⁻² s⁻¹.

In line with the above, according to one specific embodiment of the present invention the uPAR-targeting conjugate (L below) binds to the receptor (uPAR, R below) according to first order kinetics:

R+L [symbol] RL, where the reaction is characterized by the on-rate binding (Kₒₙ), the off-rate binding (K_{off}) and the resulting equilibrium binding constant K_{D} (K_{D}=K_{off}/Kₒₙ),

and wherein Kₒₙ>1×10³ M⁻¹ s⁻¹ and/or K_{off}<1×10⁻¹ s⁻¹, more preferably wherein Kₒₙ 7.3×10⁵ M⁻¹ s⁻¹.

Furthermore, according to one embodiment of the present invention, Kₒₙ of the uPAR-targeting conjugate is equal to or higher than that of uPA being the natural ligand, implying a Kₒₙ value 4.6×10⁶ M⁻¹ s⁻¹.

Moreover, also the parameters K_{D} and IC₅₀ values are measured referring to uPAR receptor binding affinity. This is further explained below in relation to the provided data.

Surface plasmon resonance (SPR) was applied to determine the IC₅₀-value of uPAR-targeting conjugate for its ability to inhibit the uPAR·uPA interaction. In brief, human recombinant uPA, which is the natural ligand for uPAR, was immobilized to the SPR sensor chip. Binding of uPAR at a concentration of 2.5 nM in the absence of UPAR-binding conjugate was examined, followed by analyses of a fixed uPAR concentration (2.5 nM) incubated with a 2-fold dilution series of uPAR-binding conjugate pre-incubated for 10 minutes at room temperature prior to injection. uPAR injection (at a rate of 50 μL/minute) was performed for 300 s (association), and dissociation was assessed for 500 s (at a rate of 50 μL/minute). The uPAR/uPAR-binding conjugate complex was injected from low to high concentration with a regeneration step followed by a buffer injection in between each injection. Regeneration was performed for 20 s by injection of 0.5 M NaCl+0.1 M Acetic acid at a 30 μL/min flow rate, followed by running buffer wash (running buffer: PBS supplemented with 0.01% Tween-20 and 50 μM EDTA).

From this type of SPR analysis, the IC₅₀ values for uPAR-targeting conjugates were determined.

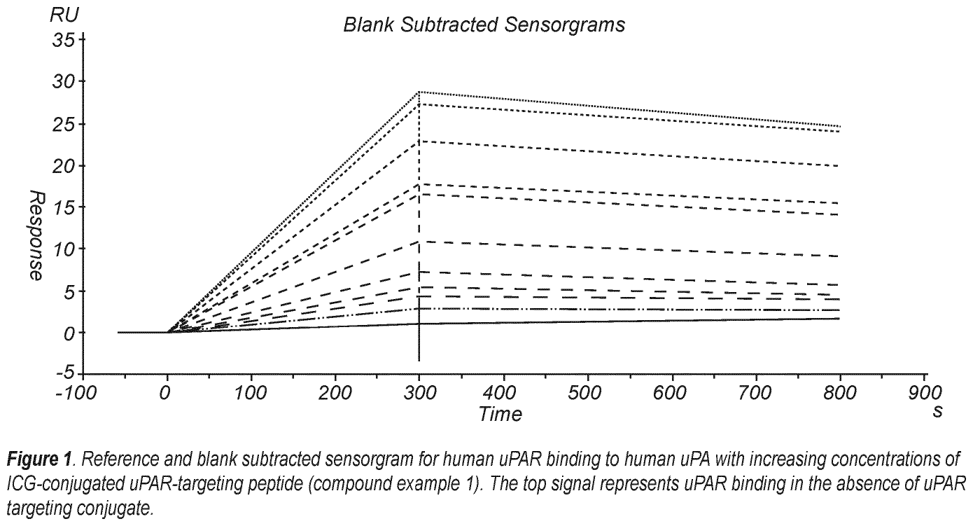

With reference to the attached figures, FIG. 1 provides reference and blank subtracted sensorgram for human uPAR binding to human uPA with increasing concentrations of ICG-conjugated uPAR-targeting peptide (compound example 1). The top signal represents uPAR binding in the absence of uPAR-targeting conjugate.

SPR was also applied to determine the K_{D}-value of binding of the uPAR-binding conjugate to uPAR. In brief, to assess the binding characteristics of uPAR-binding conjugate to uPAR, recombinant human uPAR containing a His-tag was immobilized to a NTA chip.

The uPAR-binding conjugates as well as the endogenous ligand uPA were analyzed in 2-fold dilution series from low to high concentration with a running buffer injection in between each injection. Injection of uPAR-binding conjugate or uPA (at a rate of 50 μL/minute) was performed for 120 seconds (association), and dissociation was assessed for 120 s (at a rate of 50 μL/minute). Running buffer was PBS supplemented with 0.01% Tween-20 and 50 μM EDTA).

From this assessment, the on-binding kinetics (Kₒₙ) of some uPAR-targeting conjugates were observed to be too fast and could not be determined. Therefore, the K_{D} value was determined based on steady state binding responses.

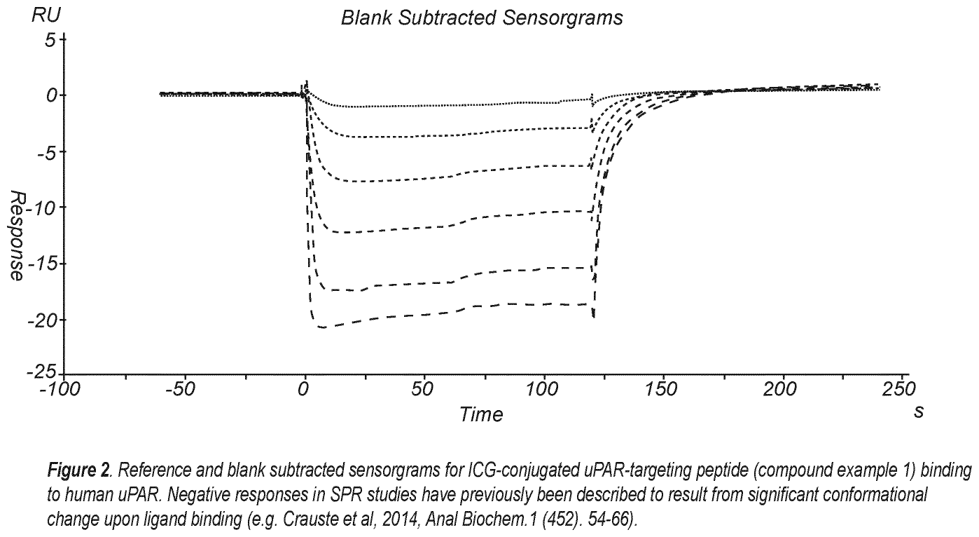

Furthermore, FIG. 2 provides reference and blank subtracted sensorgrams for ICG-conjugated uPAR-targeting peptide (compound example 1) binding to human uPAR. Negative responses in SPR studies have previously been described to result from significant conformational change upon ligand binding (e.g. Crauste et al, 2014, Anal Biochem.1 (452). 54-66).

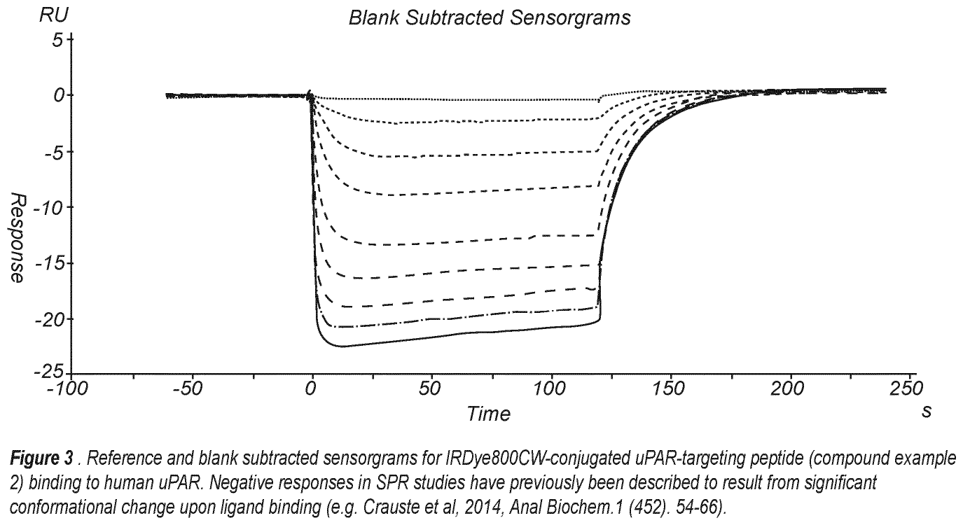

Moreover, FIG. 3 provides reference and blank subtracted sensorgrams for IRDye800CW-conjugated uPAR-targeting peptide (compound example 2) binding to human uPAR. Negative responses in SPR studies have previously been described to result from significant conformational change upon ligand binding (e.g. Crauste et al, 2014, Anal Biochem.1 (452). 54-66).

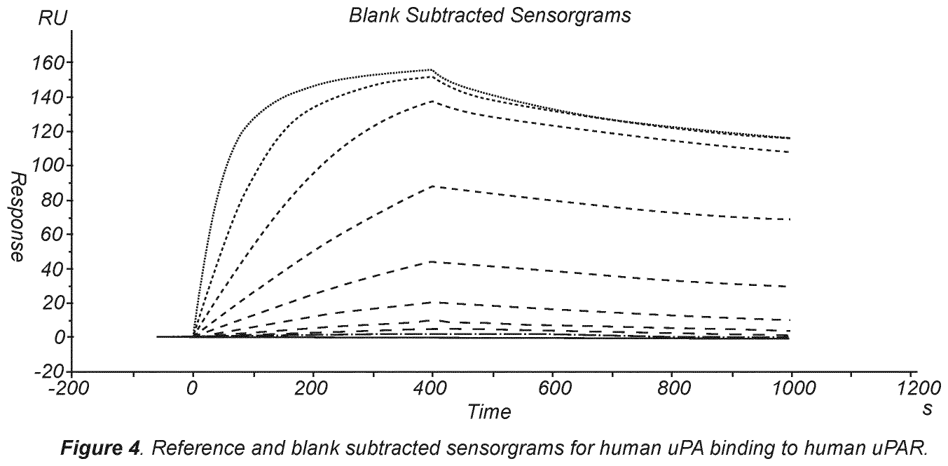

FIG. 4 shows reference and blank subtracted sensorgrams for human uPA binding to human uPAR.

Competition binding of the endogenous ligand uPA and the uPAR-targeting conjugate, e.g. human uPA and uPAR-targeting conjugate (e.g. compound example 1 according to the present invention and compound example 2 according to the present invention below) to determine IC₅₀ values (see Table 1)

Data obtained on uPAR-targeting conjugates, here uPAR targeting compound example 1 according to the present invention and compound example 2 according to the present invention is presented below in table 1. Binding affinities were determined using the above described SPR-based approach, measuring binding affinities (K_{D}) of the uPAR-targeting conjugates to immobilized human uPAR protein (the tumor target protein).

Data: Table 1

| | Compound | Compound | |
|---|---|---|---|
| | example 1 | example 2 | Human uPA |
| K_{D} (nM) | 2000 | 325 | 0.15 nM |
| Kₒₙ (M⁻¹ S⁻¹) | ≥4.6 × | ≥4.6 × | 4.6 × |
| | 10⁶ M⁻¹ s⁻¹ | 10⁶ M⁻¹ s⁻¹ | 10⁶ M⁻¹ s⁻¹ |
| IC₅₀ (nM) | 7 | 24 | ND |

Both compound examples provided above represent uPAR-targeting conjugates according to the present invention. Compound example 1 is based on the fluorophore ICG and the peptide AE105, whereas compound example 2 is based the fluorophore IRDye800CW and the peptide AE344.

Moreover, regarding plasma half-life data, the following may be mentioned. When measuring the plasma half-life the values ranged between 5.4 and 10.0 hours.

Based on this, according to one embodiment of the present invention, receptor binding affinity of the uPAR-targeting conjugate, defined as K_{D}, is maximum 2,500 nM, preferably maximum 2,000 nM, more preferably maximum 500 nM, most preferably in a range of 2,000-300 nM. Moreover, according to yet another embodiment, the uPAR-targeting conjugate displaces the natural ligand (uPA) binding to uPAR with an IC₅₀ value which is maximum 1,000 nM, preferably maximum 200 nM, more preferably maximum 50 nM, most preferably maximum 25 nM.

According to one preferred embodiment, the uPAR-targeting conjugate has a pharmacokinetic profile where an animal TBR (tumor-to-background ratio) of at least 2.5 is reached within 3.5 hours post administration and where an animal TBR of at least 2.5 is maintained at least 30 minutes before decreasing, wherein the plasma half-life is maximum 15 hours, wherein Kₒₙ of the uPAR-targeting conjugate is equal to or higher than that of uPA being the natural ligand, implying Kₒₙ≥4.6×10⁶ M⁻¹ s⁻¹, and wherein the uPAR-targeting conjugate is a human uPAR-targeting conjugate.

As is evident from above, the uPAR-targeting conjugate according to the present invention is intended to be used in cancer surgery, cancer therapy and/or cancer diagnosis. In line with this, according to one embodiment of the present invention, the uPAR-targeting conjugate according to the present invention is provided for use in cancer surgery, cancer patient risk stratification, cancer therapy or diagnosis, such as for use in optical imaging/-fluorescence imaging (FLI) of cancer. It should be said that the uPAR-targeting conjugate according to the present invention finds use in several different types of indications. Some examples are glioblastoma, glioma, primary or secondary lung cancer, colorectum cancer, breast cancer, prostate cancer, stomach cancer, gastric cancer, primary or secondary liver cancer, thyroid cancer, bladder cancer, esophagus cancer, pancreas cancer, kidney cancer, corpus uteri cancer, cervix uteri cancer, melanoma, brain (incl. central and peripheral nervous system and supporting tissue) cancer, ovary cancer, gallbladder cancer, head and neck (e.g. lip, oral cavity, larynx, nasopharynx, oropharynx, hypopharynx) cancer, multiple myeloma, testis cancer, vulva cancer, salivary glands cancer, mesothelioma cancer, penis cancer, Kaposi sarcoma, vagina cancer, neuroendocrine tumors, neuroendocrine carcinomas.

Optical imaging is of course dependent on equipment being present in the operating room. The uPAR-targeting conjugate according to the present invention contains a fluorescent chemical element that can emit light upon light excitation. The excitation and emitted light are specific to the fluorophore used. The excitation light can come from a light source, e.g. a laser such as e.g. with a wavelength between 600 nm (nano meter) and 900 nm. The emitted light from the fluorophore is typically detected by a camera using a mechanical or software-based filter e.g. detecting light between 750 nm and 950 nm. The equipment used may be a surgical robot, surgical microscope, endoscope or a handheld device. The specification of the light source (e.g. the laser or LED) and light detector (e.g. camera with filter and/or digitalized) depends on the fluorophore chosen.

Several different types of procedures may be used according to the present invention. Non-limiting examples of surgical procedures are day care surgery, open surgery, minimal invasive surgery and robot assisted surgery.

It can also be surgeries with different purpose. Non-limiting examples of surgical purposes are: Curative surgery (aims to remove all the cancerous tumor from the body—this is included into the marked size calculation), Preventive surgery (is used to remove tissue that does not contain cancerous cells but may develop into a malignant tumor, e.g. a polyps in the colon), Diagnostic surgery (helps to determine whether cells are cancerous, e.g. taking a biopsy with the aim of making a diagnostic or screenings test, such e.g. looking for colon rectal malignant polyps using a colorectal scope), Staging surgery (works to uncover the extent of cancer e.g. laparoscopy (a viewing tube with a lens or camera is inserted through a small incision to examine the inside of the body)), Debulking surgery (removes a portion, though not all, of a cancerous tumor. It is used in certain situations when removing an entire tumor may cause damage to an organ or the body), Palliative or supportive surgery (is used to treat cancer at advanced stages. It does not work to cure cancer, but to relieve discomfort or to correct other problems cancer or cancer treatment may have created. An example of supportive surgery is the insertion of a catheter to help with chemotherapy), Restorative surgery (is sometimes used as a follow-up to curative or other surgeries to change or restore a person's appearance or the function of a body part. E.g. following women with breast cancer), or Corrective surgery (is a reoperation to solve problems after surgery (or other treatments), e.g. bleedings or infection).

Furthermore, according to yet another embodiment, the present invention refers to a uPAR-targeting conjugate according to the present invention, for administration to a human body. According to yet another embodiment, the present provides a uPAR-targeting conjugate according to the present invention, for use in binding to human uPAR.

The present invention is also directed to a method intended for cancer therapy, staging or diagnosis, such as in optical imaging/fluorescence imaging (FLI) of cancer. The method may be directed to a method which involves to diagnose an anatomical structure, guide the surgeon/robot, assist the surgeon/robot, increase survival, increase the number of cancer tissues removed under surgery, increase the amount of cancer tissue removed under surgery, increase the quality of life, reduce the amount of normal tissue removed, reduce the amount of life/quality/cosmetic critical normal tissue removed, increase the certainty, reduce surgery time, improve the quality of surgery, improve the quality assurance of surgery, reduce the cost of surgery, improve the surgeon's performance, and/or improve the surgical outcome in any other way.

Furthermore, the present invention is also directed to a method involving optical imaging. Therefore, according to one specific embodiment there is disclosed an optical imaging method comprising the steps of:
(i) administering of a uPAR-targeting conjugate according to the present invention accumulating in a target tissue,
(ii) allowing time for the uPAR-targeting conjugate to accumulate in the target tissue and establishing a receptor binding, after administration into a human body;
(iii) illuminating the target tissue with light of a wavelength absorbable by the fluorophore; and
(iv) detecting fluorescence emitted by the fluorophore and forming an optical image of the target tissue.

To summarize some different aspects of the present invention, the following may be stated. The uPAR-targeting conjugate according to the present invention exhibits the following features.
A TBR is generated fast and lasts for a long time (during surgery) which is reached as a combination of:
   reaching a sufficient high concentration in plasma;
   reaching a sufficiently high concentration in the cancer tissue;
   has appropriate binding kinetics;
   has an appropriate plasma elimination half-life;
   can be dosed at dose levels which are safe, causing no severe adverse events;
   displays high selectivity to the receptor of interest that is extensively expressed on the cancer of interest, in the part of the cancer of interest and with a high selectivity to the cancer compared to normal tissue in near proximity to the cancer; and
   is detectable with available/existing equipment.

The given features above may be measured and analyzed by different means and equipment.

1. A uPAR (urokinase Plasminogen Activator Receptor)-targeting conjugate comprising:
a fluorophore;
a molecule binding to the receptor; and
a linker group which covalently links the fluorophore to the molecule binding to the receptor, said linker group either being part of the molecule binding to the receptor or being a separate component of the uPAR (urokinase Plasminogen Activator Receptor)-targeting conjugate;
wherein the uPAR (urokinase Plasminogen Activator Receptor)-targeting conjugate has a pharmacokinetic profile where a human TBR (tumor-to-background ratio) of at least 1.1 is reached within 3.5 hours post administration and where a human TBR of at least 1.5 is maintained at least 30 minutes before decreasing below 1.5, and
wherein the uPAR-targeting conjugate is a human uPAR-targeting conjugate.

2. The uPAR-targeting conjugate according to claim 1, wherein the uPAR-targeting conjugate has a pharmacokinetic profile where an animal TBR (tumor-to-background ratio) of at least 2.5 is reached within 3.5 hours post administration and where an animal TBR of at least 2.5 is maintained at least 30 minutes before decreasing, and wherein the uPAR-targeting conjugate is a human uPAR-targeting conjugate.

3. The uPAR-targeting conjugate according to claim 1, wherein the uPAR-targeting conjugate has a size of less than 50 kDa, preferably less than 20 kDa, more preferably less than 5 kDa.

4. The uPAR-targeting conjugate according to claim 1, wherein the uPAR-targeting conjugate has a size of less than 50 kDa, preferably less than 20 kDa, more preferably less than 5 kDa; and wherein the uPAR-targeting conjugate has a biological half-life in a human of at least 1 h.

5. The uPAR-targeting conjugate according to claim 1, wherein the uPAR-targeting conjugate has a pharmacokinetic profile where the plasma half-life is maximum 75 hours, preferably maximum 20 hours, more preferably maximum 15 hours, more preferably in the range of 6-15 hours, most preferably in the range of 6-10 hours.

6. The uPAR-targeting conjugate according to claim 1, wherein the uPAR-targeting conjugate has a pharmacokinetic profile where an animal TBR (tumor-to-background ratio) of at least 2.8 is reached within 3.5 hours post administration and where an animal TBR of at least 2.8 is maintained at least 30 minutes before decreasing.

7. The uPAR-targeting conjugate according to claim 1, wherein a peak animal TBR of the uPAR-targeting conjugate after administration is at least 3.

8. The uPAR-targeting conjugate according to claim 1, wherein receptor binding affinity of the uPAR-targeting conjugate to uPAR, defined as K_{D}, is maximum 2,500 nM, preferably maximum 2,000 nM, more preferably maximum 500 nM, most preferably in a range of 2,000-300 nM.

9. The uPAR-targeting conjugate according to claim 1, wherein the uPAR-targeting conjugate (L below) binds to the receptor (uPAR, R below) according to first order kinetics:
R+L [symbol] RL, where the reaction is characterized by the on-rate binding (Kₒₙ), the off-rate binding (K_{off}) and the resulting equilibrium binding constant K_{D} (K_{D}=K_{off}/Kₒₙ), and wherein Kₒₙ>1×10³ M⁻¹ s¹ and/or K_{off}<1×10⁻¹ s⁻¹, more preferably wherein Kₒₙ≥7.3×10⁵ M⁻¹ s⁻¹.

10. The uPAR-targeting conjugate according to claim 9, wherein Kₒₙ of the uPAR-targeting conjugate is equal to or higher than that of uPA being the natural ligand, implying Kₒₙ≥4.6×10⁶ M⁻¹ s⁻¹.

11. The uPAR-targeting conjugate according to claim 1, wherein the uPAR-targeting conjugate displaces the natural ligand (uPA) binding to uPAR with an IC₅₀ value which is maximum 1,000 nM, preferably maximum 200 nM, more preferably maximum 50 nM, most preferably maximum 25 nM.

12. The uPAR-targeting conjugate according to claim 1, wherein the uPAR-targeting conjugate has a sensitivity for detection of cancer tissue of at least 60%, preferably above 70%, more preferably above 80% and most preferably above 90%.

13. The uPAR-targeting conjugate according to claim 1, wherein the uPAR-targeting conjugate has a pharmacokinetic profile where an animal TBR (tumor-to-background ratio) of at least 2.5 is reached within 3.5 hours post administration and where an animal TBR of at least 2.5 is maintained at least 30 minutes before decreasing,
wherein the plasma half-life is maximum 15 hours,
wherein Kₒₙ of the uPAR-targeting conjugate is equal to or higher than that of uPA being the natural ligand, implying
Kₒₙ≥4.6×10⁶ M⁻¹ s⁻¹, and wherein the uPAR-targeting conjugate is a human uPAR-targeting conjugate.

14. A pharmaceutical composition comprising the uPAR-targeting conjugate according to claim 1, wherein the dose of the uPAR-targeting conjugate is in the range of 0.1-2,000 mg per human.

15. A uPAR-targeting conjugate according to claim 1, for administration to a human body.

16. A uPAR-targeting conjugate according to claim 1, for use in binding to human uPAR.

17. An optical imaging method comprising the steps of:
(i) administering of a uPAR-targeting conjugate according to claim 1 to a target tissue,
(ii) allowing time for the uPAR-targeting conjugate to accumulate in the target tissue and establishing a receptor binding, after administration into a human body;
(iii) illuminating the target tissue with light of a wavelength absorbable by the fluorophore; and
(iv) detecting fluorescence emitted by the fluorophore and forming an optical image of the target tissue.
